(19) United States
(12) Patent Application Publication
(10) Pub. No.: US 2021/0311213 A1
(43) Pub. Date: Oct. 7, 2021

(54) Method and System for Hybrid Positron Emission Tomography (PET) Imaging

(71) Applicant: The Board of Regents of the University of Texas System, Austin, TX (US)

(72) Inventor: Yiping Shao, Missouri City, TX (US)

(73) Assignee: The Board of Regents of the University of Texas System, Austin, TX (US)

(21) Appl. No.: 17/223,222

(22) Filed: Apr. 6, 2021

Related U.S. Application Data
(60) Provisional application No. 63/005,564, filed on Apr. 6, 2020.

(51) Int. Cl. G01T 1/29; G01T 1/161; G01T 1/202; A61B 6/00; A61B 6/03

(52) U.S. Cl. CPC G01T 1/2985; G01T 1/1617; A61B 6/5235; A61B 6/4417; A61B 6/037; G01T 1/202

(57) ABSTRACT

A method and system for generating a hybrid positron emission tomography (PET) scanner are disclosed herein. An imaging system receives, from the hybrid PET scanner, a first set of image data of an object corresponding to high-resolution, low-sensitivity image data. The imaging system receives, from the hybrid PET scanner, a second set of image data of the object corresponding to low-resolution, high-sensitivity image data. The imaging system converts the second set of image data from low-resolution, high-sensitivity image data to high-resolution, high-sensitivity image data. The imaging system combines the high-resolution, high-sensitivity image data with the high-resolution, low-sensitivity image data. The imaging system generates an image of an object based on the combined high-resolution, high-sensitivity image data and the high-resolution, low-sensitivity image data, or high-resolution, high-sensitivity image data only.

### CROSS-REFERENCE TO RELATED APPLICATIONS

This application claims priority to U.S. Provisional Application No. 63/005,564, filed Apr. 6, 2020, which is hereby incorporated by reference in its entirety.

### FIELD OF THE DISCLOSURE

This application is generally directed to a system for hybrid positron emission tomography (PET) imaging and a method for operating the same.

### BACKGROUND

High spatial resolution and high sensitivity data acquisition are important aspects for preclinical and other position emission tomography (PET) imaging applications. It is often difficult and costly to develop a PET scanner with both a high-resolution and high-sensitivity detector. Conventional approaches to such problem usually encounter an unavoidable tradeoff in performance. For example, conventional approaches may be able to obtain high-resolution but low-sensitivity or low-resolution but high-sensitivity.

### SUMMARY

In some embodiments, a method for generating a hybrid positron emission tomography (PET) image is disclosed herein. An imaging system receives, from the hybrid PET scanner, a first set of image data of an object corresponding to low-resolution, high-sensitivity image data. The imaging system receives, from the hybrid PET scanner, a second set of image data of the object corresponding to high-resolution, low-sensitivity image data. The imaging system converts the first set of image data from low-resolution, high-sensitivity image data to high-resolution, high-sensitivity image data. The imaging system combines the converted high-resolution, high-sensitivity image data with the high-resolution, low-sensitivity image data. The imaging system generates an image of an object based on either the combined converted high-resolution, high-sensitivity image data and the high-resolution, low-sensitivity image data, or the converted high-resolution, high-sensitivity image data alone.

In some embodiments, a system is disclosed herein. The system includes a hybrid positron emission tomography (PET) scanner. The hybrid PET scanner includes a hybrid detector. The system performs one or more operations. The one or more operations include receiving, from the hybrid PET scanner, a first set of image data of an object corresponding to low-resolution, high-sensitivity image data. The one or more operations further include receiving, from the hybrid PET scanner, a second set of image data of the object corresponding to high-resolution, low-sensitivity image data. The one or more operations further include converting the first set of image data from low-resolution, high-sensitivity image data to high-resolution, high-sensitivity image data. The one or more operations further include combining the high-resolution, high-sensitivity image data with the high-resolution, low-sensitivity image data. The one or more operations further include generating image of an object based on the combined high-resolution, high-sensitivity image data and the high-resolution, low-sensitivity image data, or the high-resolution, high-sensitivity image data only.

In some embodiments, a non-transitory computer readable medium is disclosed herein. The non-transitory computer readable medium has instructions stored thereon, which, when executed by a processor, cause the processor to perform an operation. The operation includes receiving, by an imaging system from a hybrid positron emission tomography (PET) scanner, a first set of image data of an object corresponding to low-resolution, high-sensitivity image data. The operation further includes receiving, by the imaging system from the hybrid PET scanner, a second set of image data of the object corresponding to high-resolution, low-sensitivity image data. The operation further includes converting, by the imaging system, the first set of image data from low-resolution, high-sensitivity image data to high-resolution, high-sensitivity image data. The operation further includes combining, by the imaging system, the high-resolution, high-sensitivity image data with the high-resolution, low-sensitivity image data. The operation further generating, by the imaging system, an image of an object based on either the combined high-resolution, high-sensitivity image data and the high-resolution, low-sensitivity image data, or the high-resolution, high-sensitivity image data only.

### BRIEF DESCRIPTION OF THE DRAWINGS

Various objectives, features, and advantages of the disclosed subject matter can be more fully appreciated with reference to the following detailed description of the disclosed subject matter when considered in connection with the following drawings, in which like reference numerals identify like elements.

FIG. 1 is a block diagram illustrating an exemplary imaging environment, according to example embodiments.

FIG. 2A is a block diagram illustrating a hybrid detector from the exemplary imaging environment of FIG. 1, according to example embodiments.

FIG. 2B is a block diagram illustrating a hybrid detector from the exemplary computing environment of FIG. 1, according to example embodiments.

FIG. 2C is a block diagram illustrating a hybrid detector from the exemplary imaging environment of FIG. 1, according to example embodiments.

FIG. 2D is a block diagram illustrating a hybrid detector from the exemplary computing environment of FIG. 1, according to example embodiments.

FIG. 3 is a flow chart illustrating a method of imaging a patient, according to example embodiments.

FIG. 4A illustrates a system bus imaging system architecture, according to example embodiments.

FIG. 4B illustrates a computer system having a chipset architecture, according to example embodiments.

FIG. 5A illustrates a simulated whole body-positron emission tomography, according to example embodiments.

FIG. 5B illustrates reconstructed images and line profiles, according to example embodiments.

The drawings are not necessarily to scale, or inclusive of all elements of a system, emphasis instead generally being placed upon illustrating the concepts, structures, and techniques sought to be protected herein.

### DETAILED DESCRIPTION

High-resolution and high-sensitivity data acquisition are both important for preclinical and other PET imaging applications. In conventional PET designs, however, to achieve both high-resolution and high-sensitivity data acquisition at the same time is challenging, because it is technically difficult to develop both high-resolution and high-sensitivity data acquisition with small and long scintillator crystals. Such data acquisition is even more demanding if depth-of-interaction (DOI) is required. As a result, the cost of such detector can be very high. Further, due to the performance tradeoff, it is usually unavoidable to focus on the high-resolution data acquisition with thin and small pixel scintillators that can be relatively easy to develop at low cost even with lower sensitivity and deteriorated image performance. Conventional systems have attempted to address this problem by developing a dedicated high-resolution PET. Such attempts are difficult and expensive to develop.

In addition, for an existing PET, such as a whole-body PET, its resolution is not sufficient for high-resolution imaging applications, such as for a dedicated brain imaging.

The one or more techniques disclosed herein address the high-resolution and high-sensitivity data acquisition problem through the use of a hybrid scanner. The hybrid scanner may include two sub-detectors: a first sub-detector that may be a high-resolution, low-sensitivity detector with small and short scintillator arrays; and a second sub-detector that may be a low-resolution, high-sensitivity detector with large and long scintillator arrays. Through the use of two sub-detectors, the cost and development technical difficulty may be reduced and controlled, or the resolution of an existing PET can be improved.

To obtain both a high-resolution and high-sensitivity image, the data sets obtained by both the sub-detectors may be provided to a hybrid imaging system. The hybrid imaging system may be configured to convert the data acquired from the second sub-detector (i.e., the low-resolution, high-sensitivity sub-detector) to equivalent high-resolution, high-sensitivity data for improving the final high-resolution image while still maintain the high-sensitivity.

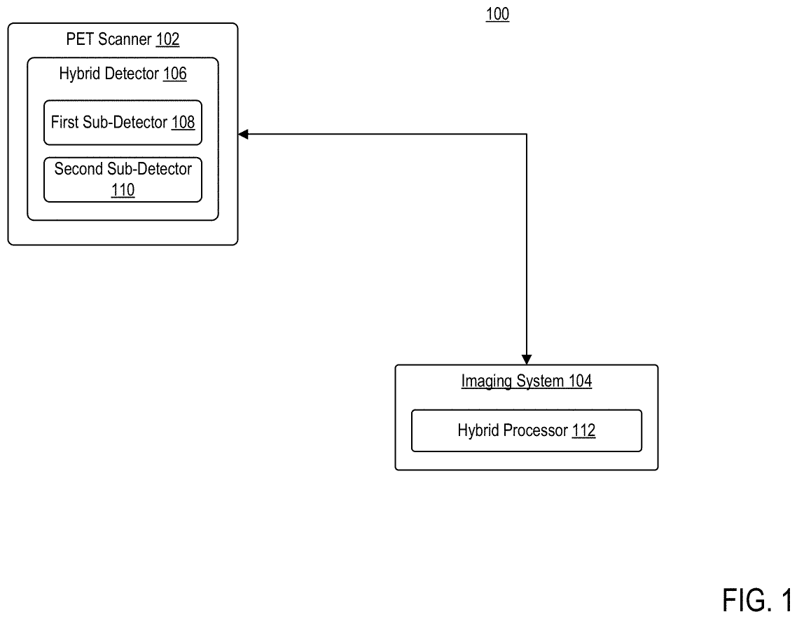

FIG. 1 is a block diagram illustrating an exemplary computing environment 100, according to example embodiments. Computing environment 100 may include PET scanner 102 in communication with imaging system 104. In some embodiments, PET scanner 102 may be in communication with imaging system 104 via wired connection. In some embodiments, PET scanner 102 may be in communication with imaging system 104 via signal transfer, such as over one or more computing networks.

PET scanner 102 may represent an apparatus configured to capture one or more images of a patient. For example, PET scanner 102 may be configured to capture one or more dynamic images of internal organs and/or tissues of a given patient. PET scanner 102 may include hybrid detector 106. Hybrid detector 106 may be configured to capture imaging data through the imaging acquisition process. Hybrid detector 106 may include, for example, a first sub-detector 108 and a second sub-detector 110. Each sub-detector 108, 110 may be focused and optimized for either high-resolution or high-sensitivity. The first sub-detector 108 may be physically combined with the second sub-detector 110, or the first sub-detector may not be physically combined with the second sub-detector 110.

First sub-detector 108 may be configured as a high-resolution, low-sensitivity detector. For example, first sub-detector 108 may be formed from one or more small and short scintillator arrays. In some embodiments, first sub-detector 108 may be formed from a 32×32 array of 1×1×3 mm³ lutetium yttrium orthosilicate (LYSO) scintillators. First sub-detector 108 may be configured to capture high-resolution lines of response (HH LORs). Second sub-detector 110 may be configured as a low-resolution, high-sensitivity detector. For example, second sub-detector 110 may be formed from one or more large and long scintillator arrays. In some embodiments, second sub-detector 110 may be formed from an 8×8 array of 4×4×17 mm³ LYSO scintillators. Second sub-detector 110 may be configured to capture low-resolution lines of response (LL LORs). Hybrid detector 106 may transmit data associated with HH LORs and LL LORs to imaging system 104 for processing.

Imaging system 104 may be in communication with PET scanner 102 via one or more wired and/or wireless connections. Imaging system 104 may be operated by a user. For example, imaging system 104 may be a mobile device, a tablet, a desktop computer, or any imaging system having the capabilities described herein.

Imaging system 104 may include at least hybrid processor 112. Hybrid processor 112 may be configured to receive the data associated with HH LORs and LL LORs from PET scanner 102. Upon receiving the data, hybrid processor 112 may be configured to convert the received low-resolution data into high-resolution data, thereby achieving both high-resolution and high-sensitivity data for hybrid imaging.

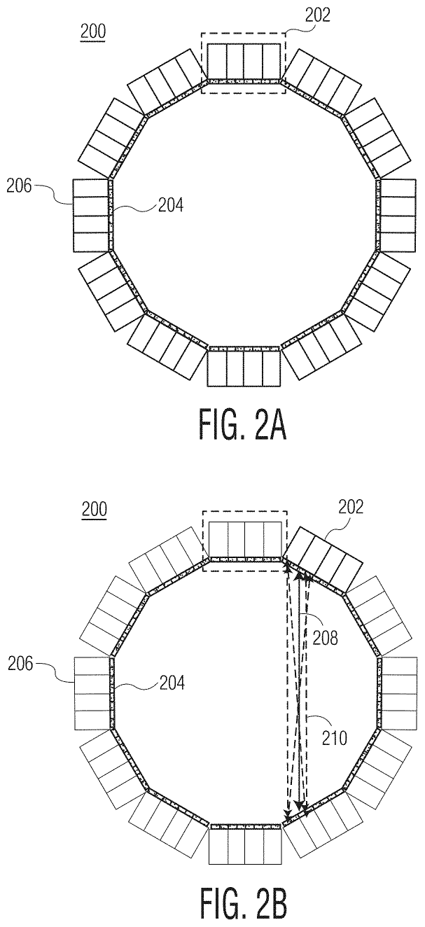

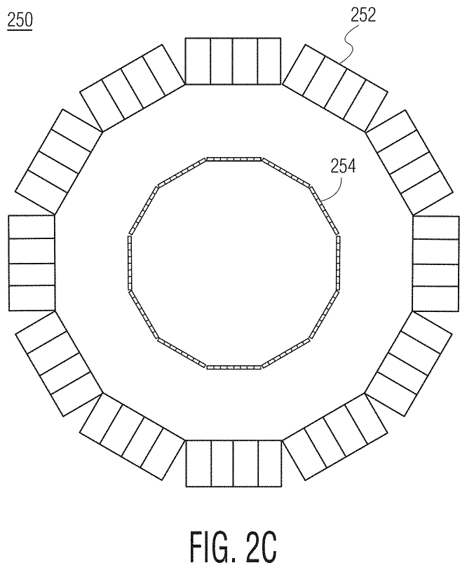

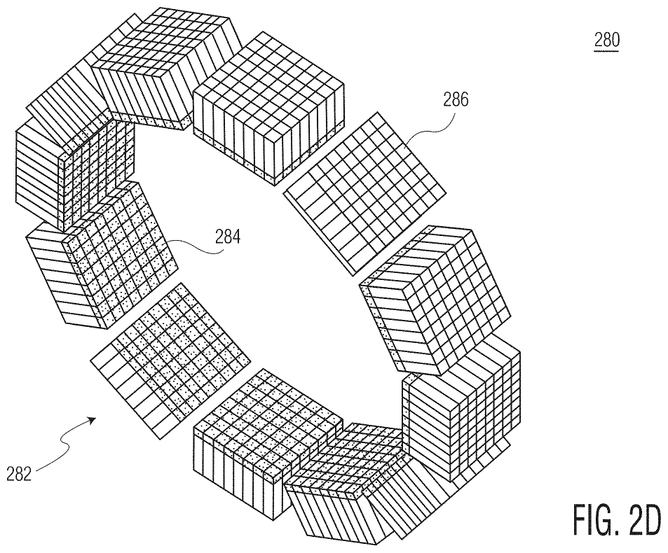

Generally, for a coincidence projection i (illustrated in FIG. 2B), PET Scanner 102 may receive counts to HH and LL₁, LL₂, . . . , LLₙ, where 1 n may indicate the possible total LL LORs. Mathematically, if LL₁ᵢ is part of the LL₁ contributed from projection i, and similarly for LL₂ᵢ to LLₙᵢ and Nᵢ may represent the number of summed LL₁ᵢ to LLₙᵢ, which provides:

LL₁ᵢ=P₁ᵢ(N)ᵢ,LL₂ᵢ=P₂ᵢ(N)ᵢ, . . . LLₙᵢ=Pₙᵢ(N)ᵢ

Or, equivalently,

(LL)₁ᵢ=Pⱼᵢ(N)ᵢ, j=1 . . . n (1)

where Pⱼᵢ represents the probability of one count of (N)ᵢ generating one count of LLⱼᵢ, and the sum of P₁ᵢ to Pₙᵢ is equal to 1, i.e.,

Σⱼ₌₁ⁿPⱼᵢ=1, (2)

Extending Eq. (1) to include all projects, may yield:

$$( \begin{matrix}L {L}_{1} \\ L {L}_{2} \\ … \\ L {L}_{n}\end{matrix} ) = ( \begin{matrix}{P}_{11} & {P}_{12} & … & {P}_{1 n} \\ {P}_{21} & {P}_{22} & … & {P}_{2 n} \\ … & … & … & … \\ {P}_{m 1} & {P}_{m 2} & … & {P}_{m n}\end{matrix} ) ( \begin{matrix}{N}_{1} & {N}_{1} & … & {N}_{1} \\ {N}_{2} & {N}_{2} & … & {N}_{2} \\ … & … & … & … \\ {N}_{m} & {N}_{m} & … & {N}_{m}\end{matrix} )$$

Or, equivalently,

$$L {L}_{j} = {\sum }_{i = 1}^{m} {{P}_{j i} ( N )}_{i} , \qquad ( 3 )$$

where m is the total number of projections, and where m>n.

Eq. 3 is mathematically similar to a forward projection used in iterative image reconstruction, where (N)ᵢ may be the coincidence interactions that generate the “projected” data LL₁ by second sub-detectors 110. Therefore, (N)ᵢ may be considered as the “source,” LL₁ as the acquired “projection data,” and Pⱼᵢ as the “system matrix.”

Hybrid processor 112 may be configured to solve Eq. 3 to obtain the “source” (N)ᵢ, which may then be used as the high-resolution LORs. In some embodiments, Hybrid processor 112 may solve the inverse problem using a maximum likelihood—expectation maximization (ML-EM) iterative method, which may be expressed as:

$${N}_{i}^{s + 1} = \frac{{N}_{i}^{s}}{{Σ}_{j} {P}_{j i}} {\sum }_{j} {P}_{j i} \frac{L {L}_{j}}{{Σ}_{k} {P}_{j k} {N}_{k}^{s}}$$

In some embodiments, Hybrid processor 112 may use the acquired high-resolution data, HHᵢ, that contains the detected high-resolution data by the first sub-detector as the prior information for the initial Nᵢ⁰ to improve the efficiency and accuracy of the data conversion. Although in general this is not always required.

If the conversion matrix, Pⱼᵢ, is known, hybrid processor 112 may be able to convert the projection data acquired from second sub-detector 110 (i.e., the low-resolution data) to equivalent high-resolution data, Nᵢ^{final} for substantially improving the image resolution. If necessary, hybrid processor may also combine all HHᵢ and Nᵢ^{final} together for further increase the sensitivity.

In other words, hybrid processor 112 may be configured to generate a conversion matrix for hybrid detector 106, such that when hybrid processor 112 receives data from PET scanner 102, hybrid processor 112 can easily and efficiently convert the data acquired from second sub-detector 110 (i.e., the low-resolution, high-sensitivity sub-detector) to equivalent high-resolution data. As such, hybrid processor 112 can achieve both high-resolution and high-sensitivity imaging data from hybrid detector 106.

FIG. 2A is a block diagram illustrating a hybrid detector 200 from the exemplary computing environment 100 of FIG. 1, according to example embodiments. Hybrid detector 200 may correspond to hybrid detector 106 discussed above.

As illustrated, hybrid detector 200 may include at least plurality of detector units 202. For example, as illustrated the plurality of detector units may be arranged in an array. Each detector unit 202 may include a high-resolution sub-detector 204 and a low-resolution sub-detector 206. High-resolution sub-detector 204 may be formed from one or more small and short scintillator arrays. Generally, high-resolution sub-detector 204 may be configured to capture high-resolution lines of response. Low-resolution sub-detector 206 may be formed from one or more large and long scintillator arrays. Low-resolution sub-detector 206 may be configured to capture low-resolution lines of responses.

As shown, high-resolution sub-detector 204 may be positioned interior to low-resolution sub-detector 206. For example, high-resolution sub-detector 204 may be configured to face an imaging object.

FIG. 2B is a block diagram illustrating hybrid detector 200 from the exemplary imaging environment of FIG. 1, according to example embodiments. As illustrated in FIG. 2B, one or more coincident gamma rays 208 and one or more coincident projections 210 are shown extending between two detector units 202.

FIG. 2C is a block diagram illustrating a hybrid detector 250 from the exemplary imaging environment of FIG. 1, according to example embodiments. Hybrid detector 250 may correspond to hybrid detector 106 discussed above.

As illustrated, hybrid detector 250 may include first array of low-resolution detector units 252 and a second array of high-resolution detector units 254. In some embodiments, first array of low-resolution detector units 252 may be spaced from second array of high-resolution detector units 254. Each high-resolution detector 254 may be formed from one or more small and short scintillator arrays. Generally, high-resolution sub-detector 254 may be configured to capture high-resolution lines of response. Each low-resolution detector 252 may be formed from one or more large and long scintillator arrays. Low-resolution sub-detector 252 may be configured to capture low-resolution lines of responses.

As shown, second array of high-resolution detector units 254 may be positioned interior to first array of low-resolution detector units 252. For example, second array of high-resolution detector units 254 may be configured to face an imaging object. In some embodiments, first array of low-resolution detector units 252 may have a diameter between about 70 to 100 cm, such as a clinical whole-body PET. In some embodiments, second array of high-resolution detector units 254 may have a diameter between about 30-45 cm for human brain imaging. In some embodiments, the second array of high-resolution detector units 254 may be configured as an insert device, which can be inserted inside the first array of low-resolution detector units 252 for generating combined high-resolution and low-resolution data from two sub-detectors that can be used to generate conversion matrix, or be removed outside the first array of low-resolution detector units 252 for only acquiring low-resolution line-of-responses which can be converted to high-resolution line-of-responses with the known conversion matrix.

FIG. 2D is a block diagram illustrating a hybrid detector 280 from the exemplary computing environment 100 of FIG. 1, according to example embodiments. Hybrid detector 280 may correspond to hybrid detector 106 discussed above.

As illustrated, hybrid detector 280 may include at least plurality of detector units 282. For example, as illustrated the plurality of detector units may be arranged in an array. Each detector unit 282 may include a high-resolution sub-detector 284 and a low-resolution sub-detector 286. High-resolution sub-detector 284 may be formed from one or more small and short scintillator arrays. Generally, high-resolution sub-detector 284 may be configured to capture high-resolution lines of response. Low-resolution sub-detector 286 may be formed from one or more large and long scintillator arrays. Low-resolution sub-detector 286 may be configured to capture low-resolution lines of responses.

As shown, hybrid detector 280 may include twelve detector units 282. Each high-resolution sub-detector 284 may include a 32×32 array of 1×1×3 mm³ LYSO scintillators. Each low-resolution sub-detector 286 may include 4×4×17 mm³ LYSO scintillators. Low-resolution sub-detectors 286 may be placed on top of high-resolution sub-detectors 284.

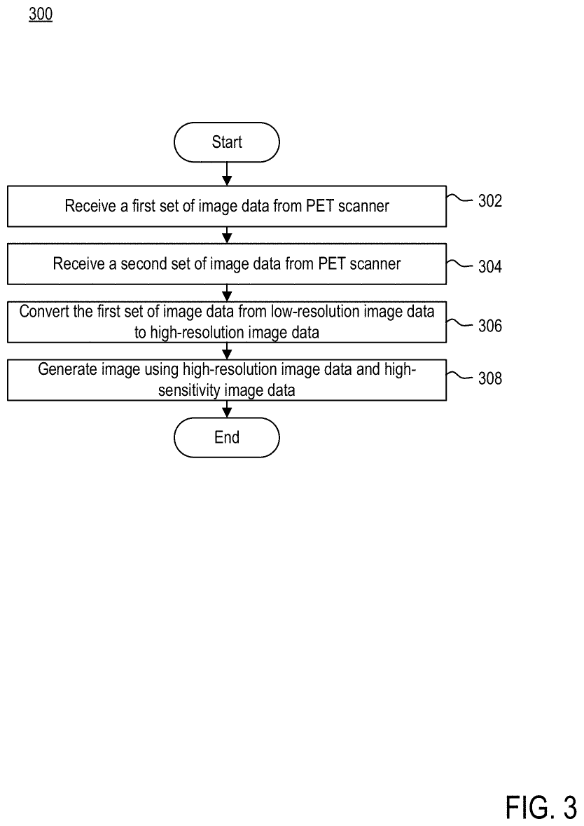

FIG. 3 is a flow chart illustrating a method 300 of imaging an object using PET scanner 102, according to example embodiments. Method 300 may begin at step 302.

At step 302, imaging system 104 may receive a first set of image data from PET scanner 102. First set of image data may be captured using first sub-detector 108 of PET scanner 102. For example, first sub-detector 108 may be configured to capture high-resolution, low-sensitivity image data of an object. In some embodiments, first sub-detector 108 may be formed from a 32×32 array of 1×1×3 mm³ LYSO scintillators.

At step 304, imaging system 104 may receive a second set of image data from PET scanner 102. Second set of image data may be captured using second sub-detector 110 of PET scanner 102. For example, second sub-detector 110 may be configured to capture low-resolution, high-sensitivity image data for an object. In some embodiments, second sub-detector 110 may be formed from an 8×8 array of 4×4×17 mm³ LYSO scintillators.

At step 306, imaging system 104 may convert the image data received from the second sub-detector 110 from low-resolution image data to high-resolution image data. For example, hybrid processor 112 may be configured to generate a conversion matrix for hybrid detector 106, given data related to first sub-detector 108 and second sub-detector 110 in a “conversion matrix generation procedure” prior to the patient imaging procedure. Using the conversion matrix, hybrid processor 112 may convert the data acquired from second sub-detector 110 (i.e., the low-resolution, high-sensitivity sub-detector) in the patient imaging procedure to equivalent high-resolution data. As such, Hybrid processor 112 can achieve both high-resolution and high-sensitivity imaging data from hybrid detector 106.

At step 308, imaging system 104 may generate an image using only the converted high-resolution and high-sensitivity imaging data from the low-resolution, high-sensitivity imaging data generated by the second sub-detector 110 of hybrid detector 106, or using combined the converted high-resolution, high-sensitivity imaging data and the high-resolution, low-sensitivity imaging data generated by the first sub-detector 108 of hybrid detector 106.

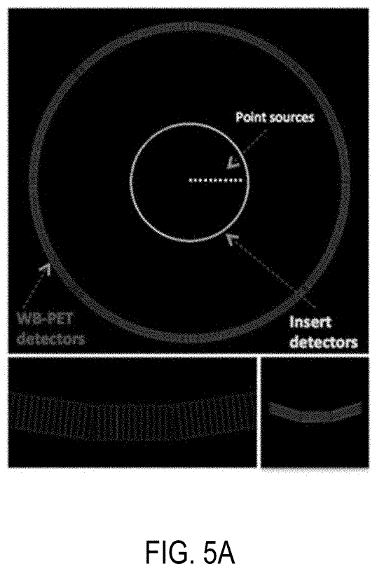

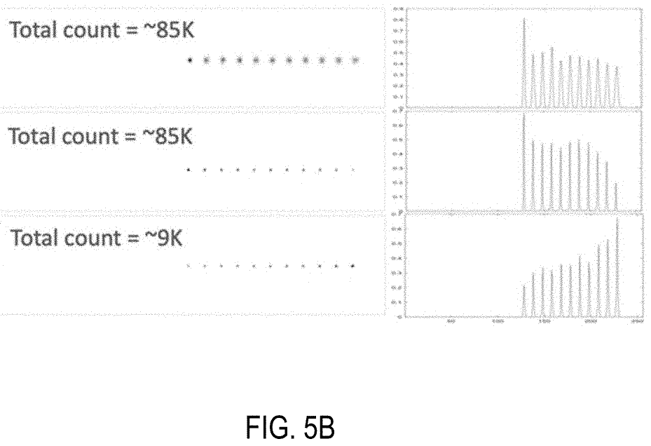

To confirm the results, a simulation study was conducted to test whether the data conversion process of hybrid (whole body (WB)-PET) imaging would work with two-dimensional point source images. For the simulation, a GATE simulation package to simulate a general WB-PET, an insert detector ring, and point sources at different off center field-of-view positions were used, as shown in FIG. 5A. In some embodiments, for the simulation, the WB-PET detector has an 84.2 cm inner diameter and a 22.1 cm axial length. The WB-PET detector may include four detector rings, each with 48 detector units. Each unit may include of a 13×13 array of 4×4×20 mm³ LYSO scintillators, with 0.24 mm inter-scintillator gaps. In some embodiments, for the simulation study, the insert detector ring has a 32.0 cm diameter and a 10.5 cm axial length. The insert detector ring may also include 48 detector units. Each unit may include an 18×18 array of 1×1×3 mm³ LYSO scintillators, with 0.15 mm inter-scintillator gaps. In some embodiments, for the simulation study, eleven point sources were placed along a line with different field-of-view positions, ranging from 0.0 to 10.0 cm off-center distances, with 1.0 cm gaps between the neighboring point sources.

In some embodiments, maximum likelihood expectation maximization image reconstruction was used for the simulation. FIG. 5B shows the point source images reconstructed from the data of the WB-PET imaging, the hybrid WB-PET imaging, and the imaging from a high-resolution WB-PET with 1.0 mm image resolution as a gold standard reference for comparison. In some embodiments, for the simulation study, the spatial resolutions (full width at half maximum) measured from these simulated images are 2.79±0.43, 1.06±0.06 and 1.02±0.03 mm, respectively. The corresponding counts used in image recon are 85324, 85324 and 8916. The results show that the hybrid WB-PET can substantially improve the image resolution of a WB-PET to the level that is comparable to the image resolution of a high-resolution PET (such high-resolution PET would be very difficult and expensive to develop).

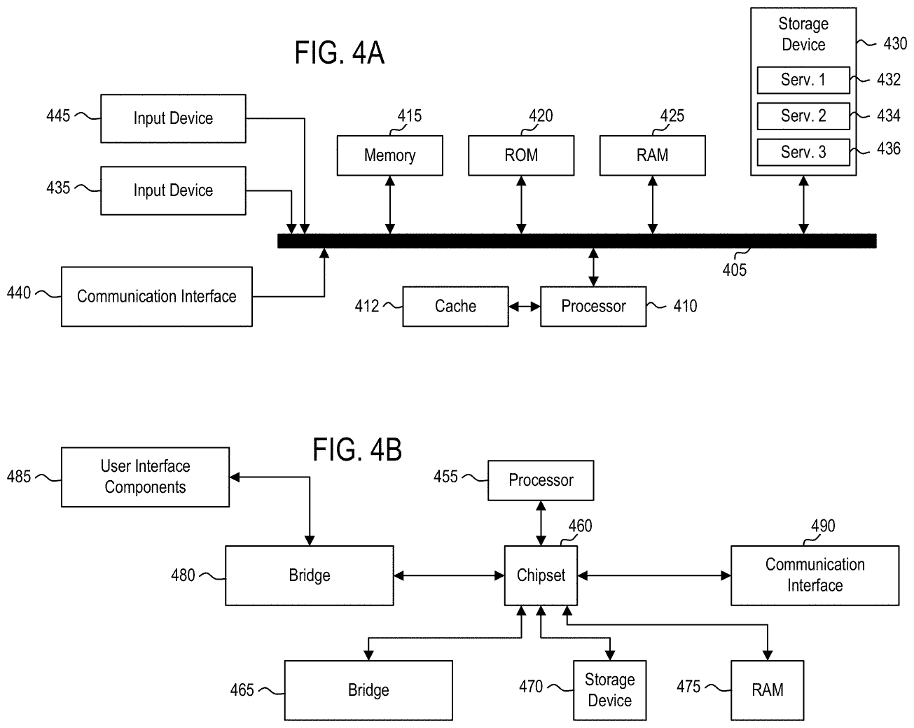

FIG. 4A illustrates a system bus imaging system architecture 400, according to example embodiments. System 400 may be representative of an imaging system capable of performing the functions described above. One or more components of system 400 may be in electrical communication with each other using a bus 405. System 400 may include a processing unit (CPU or processor) 410 and a system bus 405 that couples various system components including the system memory 415, such as read only memory (ROM) 420 and random access memory (RAM) 425, to processor 410. System 400 may include a cache of high-speed memory connected directly with, in close proximity to, or integrated as part of processor 410. System 400 may copy data from memory 415 and/or storage device 430 to cache 412 for quick access by processor 410. In this way, cache 412 may provide a performance boost that avoids processor 410 delays while waiting for data. These and other modules may control or be configured to control processor 410 to perform various actions. Other system memory 415 may be available for use as well. Memory 415 may include multiple different types of memory with different performance characteristics. Processor 410 may include any general purpose processor and a hardware module or software module, such as service 1432, service 2434, and service 3436 stored in storage device 430, configured to control processor 410 as well as a special-purpose processor where software instructions are incorporated into the actual processor design. Processor 410 may essentially be a completely self-contained imaging system, containing multiple cores or processors, a bus, memory controller, cache, etc. A multi-core processor may be symmetric or asymmetric.

To enable user interaction with the computing device 400, an input device 445 may represent any number of input mechanisms, such as a microphone for speech, a touch-sensitive screen for gesture or graphical input, keyboard, mouse, motion input, speech and so forth. An output device 435 may also be one or more of a number of output mechanisms known to those of skill in the art. In some instances, multimodal systems may enable a user to provide multiple types of input to communicate with computing device 400. Communications interface 440 may generally govern and manage the user input and system output. There is no restriction on operating on any particular hardware arrangement and therefore the basic features here may easily be substituted for improved hardware or firmware arrangements as they are developed.

Storage device 430 may be a non-volatile memory and may be a hard disk or other types of computer readable media which may store data that are accessible by a computer, such as magnetic cassettes, flash memory cards, solid state memory devices, digital versatile disks, cartridges, random access memories (RAMs) 425, read only memory (ROM) 420, and hybrids thereof.

Storage device 430 may include services 432, 434, and 436 for controlling the processor 410. Other hardware or software modules are contemplated. Storage device 430 may be connected to system bus 405. In one aspect, a hardware module that performs a particular function may include the software component stored in a computer-readable medium in connection with the necessary hardware components, such as processor 410, bus 405, display 435, and so forth, to carry out the function.

FIG. 4B illustrates a computer system 450 having a chipset architecture. Computer system 450 may be an example of computer hardware, software, and firmware that may be used to implement the disclosed technology. System 450 may include a processor 455, representative of any number of physically and/or logically distinct resources capable of executing software, firmware, and hardware configured to perform identified computations. Processor 455 may communicate with a chipset 460 that may control input to and output from processor 455. In this example, chipset 460 outputs information to output 465, such as a display, and may read and write information to storage device 470, which may include magnetic media, and solid state media, for example. Chipset 460 may also read data from and write data to RAM 475. A bridge 480 for interfacing with a variety of user interface components 485 may be provided for interfacing with chipset 460. Such user interface components 485 may include a keyboard, a microphone, touch detection and processing circuitry, a pointing device, such as a mouse, and so on. In general, inputs to system 450 may come from any of a variety of sources, machine generated and/or human generated.

Chipset 460 may also interface with one or more communication interfaces 490 that may have different physical interfaces. Such communication interfaces may include interfaces for wired and wireless local area networks, for broadband wireless networks, as well as personal area networks. Some applications of the methods for generating, displaying, and using the GUI disclosed herein may include receiving ordered datasets over the physical interface or be generated by the machine itself by processor 455 analyzing data stored in storage 470 or 475. Further, the machine may receive inputs from a user through user interface components 485 and execute appropriate functions, such as browsing functions by interpreting these inputs using processor 455.

It may be appreciated that example systems 400 and 450 may have more than one processor 410 or be part of a group or cluster of computing devices networked together to provide greater processing capability.

While the foregoing is directed to embodiments described herein, other and further embodiments may be devised without departing from the basic scope thereof. For example, aspects of the present disclosure may be implemented in hardware or software or a combination of hardware and software. One embodiment described herein may be implemented as a program product for use with a computer system. The program(s) of the program product define functions of the embodiments (including the methods described herein) and can be contained on a variety of computer-readable storage media. Illustrative computer-readable storage media include, but are not limited to: (i) non-writable storage media (e.g., read-only memory (ROM) devices within a computer, such as CD-ROM disks readably by a CD-ROM drive, flash memory, ROM chips, or any type of solid-state non-volatile memory) on which information is permanently stored; and (ii) writable storage media (e.g., floppy disks within a diskette drive or hard-disk drive or any type of solid state random-access memory) on which alterable information is stored. Such computer-readable storage media, when carrying computer-readable instructions that direct the functions of the disclosed embodiments, are embodiments of the present disclosure.

It will be appreciated to those skilled in the art that the preceding examples are exemplary and not limiting. It is intended that all permutations, enhancements, equivalents, and improvements thereto are apparent to those skilled in the art upon a reading of the specification and a study of the drawings are included within the true spirit and scope of the present disclosure. It is therefore intended that the following appended claims include all such modifications, permutations, and equivalents as fall within the true spirit and scope of these teachings.

What is claimed is:

1. A method for generating a hybrid positron emission tomography (PET) scanner, comprising:
receiving, by an imaging system from the hybrid PET scanner, a first set of image data of an object corresponding to high-resolution, low-sensitivity image data;
receiving, by the imaging system from the hybrid PET scanner, a second set of image data of the object corresponding to low-resolution, high-sensitivity image data;
converting, by the imaging system, the second set of image data from low-resolution, high-sensitivity image data to high-resolution, high-sensitivity image data;
combining, by the imaging system, the high-resolution, high-sensitivity image data with the high-resolution, low-sensitivity image data; and
generating, by the imaging system, an image of an object based on either the combined high-resolution, high-sensitivity image data and the high-resolution, low-sensitivity image data, or the high-resolution, high-sensitivity image data only.

2. The method of claim 1, wherein the hybrid PET scanner comprises:
a hybrid detector comprising a first sub-detector and a second sub-detector.

3. The method of claim 2, wherein the first set of image data is received from the first sub-detector and the second set of image data is received from the second sub-detector.

4. The method of claim 3, wherein the first sub-detector comprises one or more 32×32 array of 1×1×3 mm³ LYSO scintillators, or array of small cross-sectional area and short scintillators.

5. The method of claim 3, wherein the second sub-detector comprises one or more 8×8 array of 4×4×17 mm³ LYSO scintillators, or array of large cross-sectional area and long scintillators.

6. The method of claim 2, further comprising:
generating, by the imaging system, a conversion matrix based on a configuration of the first sub-detector and the second sub-detector.

7. The method of claim 6, wherein the conversion matrix is based on projection data between the first sub-detector and the second sub-detector.

8. A system, comprising:
a processor in communication with a hybrid positron emission tomography (PET) scanner comprising a hybrid detector; and
a memory having programming instructions stored thereon, which when executed by the processor, performs one or more operations comprising:
receiving, by a imaging system from the hybrid PET scanner, a first set of image data of an object corresponding to high-resolution, low-sensitivity image data;
receiving, by the imaging system from the hybrid PET scanner, a second set of image data of the object corresponding to low-resolution, high-sensitivity image data;
converting, by the imaging system, the second set of image data from low-resolution, high-sensitivity image data to high-resolution, high-sensitivity image data;
combining, by the imaging system, the high-resolution, high-sensitivity image data with the high-resolution, low-sensitivity image data; and
generating, by the imaging system, an image of an object based on either the combined high-resolution, high-sensitivity image data and the high-resolution, low-sensitivity image data, or the high-resolution, high-sensitivity image data only.

9. The system of claim 8, wherein the hybrid PET scanner comprises:
a hybrid detector comprising a first sub-detector and a second sub-detector.

10. The system of claim 9, wherein the first set of image data is received from the first sub-detector and the second set of image data is received from the second sub-detector.

11. The system of claim 10, wherein the first sub-detector comprises one or more 32×32 array of 1×1×3 mm³ LYSO scintillators, or arrays of small cross-sectional area and short scintillators.

12. The system of claim 10, wherein the second sub-detector comprises one or more 8×8 array of 4×4×17 mm³ LYSO scintillators, or arrays of large cross-sectional area and long scintillators.

13. The system of claim 9, wherein the one or more operations further comprise:
generating, by the imaging system, a conversion matrix based on a configuration of the first sub-detector and the second sub-detector.

14. The system of claim 13, wherein the conversion matrix is based on projection data between the first sub-detector and the second sub-detector.

15. A non-transitory computer readable medium having instructions stored thereon, which, when executed by a processor, cause the processor to perform an operation, comprising:
receiving, by a imaging system from a hybrid positron emission tomography (PET) scanner, a first set of image data of an object corresponding to high-resolution, low-sensitivity image data;
receiving, by the imaging system from the hybrid PET scanner, a second set of image data of the object corresponding to low-resolution, high-sensitivity image data;
converting, by the imaging system, the second set of image data from low-resolution, high-sensitivity image data to high-resolution, high-sensitivity image data;
combining, by the imaging system, the high-resolution, high-sensitivity image data with the high-resolution, low-sensitivity image data; and
generating, by the imaging system, an image of an object based on the combined high-resolution, high-sensitivity image data and the high-resolution, low-sensitivity image data, or based on the converted high-resolution, high-sensitivity image data only.

16. The non-transitory computer readable medium of claim 15, wherein the hybrid PET scanner comprises:
a hybrid detector comprising a first sub-detector and a second sub-detector.

17. The non-transitory computer readable medium of claim 16, wherein the first set of image data is received from the first sub-detector and the second set of image data is received from the second sub-detector.

18. The non-transitory computer readable medium of claim 17, wherein the first sub-detector comprises one or more 32×32 array of 1×1×3 mm³ LYSO scintillators, or arrays of small cross-sectional area and short scintillators.

19. The non-transitory computer readable medium of claim 17, wherein the second sub-detector comprises one or more 8×8 array of 4×4×17 mm³ LYSO scintillators, or arrays of large cross-sectional area and long scintillators.

20. The non-transitory computer readable medium of claim 16, wherein the operation further comprises:
generating, by the imaging system, a conversion matrix based on a configuration of the first sub-detector and the second sub-detector.
